(19) United States
(12) Patent Application Publication
(10) Pub. No.: US 2008/0255586 A1
(43) Pub. Date: Oct. 16, 2008

(54) Assembly for Use in Dermabrasion Having an Abrasive Disc

(76) Inventor: Allan Ronald Greenberg, South Australia (AU)

Correspondence Address: Francis Law Group, 1942 Embarcadero, Oakland, CA, 94606, US

(21) Appl. No.: 11/631,803

(22) PCT Filed: Jul. 6, 2005
(86) PCT No.: PCT/AU2005/000989

§ 371 Date: Jan. 8, 2008

(30) Foreign Application Priority Data
Jul. 7, 2004 (AU) 2004903701

(51) Int. Cl. A61B 17/50

(52) U.S. Cl. 606/131

(57) ABSTRACT

The present invention relates to an assembly having a handle, cap and abrasive disc for use in dermabrasion. The assembly is able to be used to vigorously abrade the skin when said abrasive disc protrudes beyond and through an opening in said cap. The abrasive disc can then be retracted or repositioned within a chamber enclosed by said cap and the tool can be used to draw off the dead skin particles or gently abraded the skin surface. The tool of the present invention has the ability to be adjusted to the specific requirements of the user as the same handle allows the attachment of variously sized abrasive discs and caps to facilitate treatment.

### FIELD OF THE INVENTION

The present invention relates to an assembly for use in dermabrasion having an abrasive disc.

### BACKGROUND OF THE INVENTION

Dermabrasion of the skin by the use of abrasive material is a known technique. Human skin is composed of three integrated layers, the epidermis, dermis and subcutis. The bottom of the epidermis is where cell division generates new cells that travel up through the epidermis until they become the upper surface layers after some 3-4 weeks. The outermost layer of the epidermis is composed of several layers of dead skin, which act as a buffer to the lower layers of skin.

As a person ages the epidermis begins to degenerate becoming both thicker and dryer. This can lead to blocked pores and other skin diseases. Skin damage from the sun and other sources also accumulates with age and further affects the appearance of the skin. As a result, various treatments, including daily cleaning and facial scrubs, are used to reduce the signs of aging and to ensure that the skin remains healthy and clean.

A number of devices have been proposed to assist in removing dead skin cells from the epidermis, including scraping blades and scrubbing pads. These devices dislodge the dead skin cells, but do not remove them from the skin surface. Consequently, unless the area is thoroughly cleaned, the dead skin cells can remain on the skin surface and may result in blocked pores.

Dermabrasion treatments are also used for the mitigation of scar tissue. Abrasive pads and chemicals are used to reduce the appearance of scars that result from surgery or accidents. These types of treatments are often used where the scar tissue is in a prominent position, such as on the face or arms and is a particularly useful treatment when sun spots and lesions have been removed. Dermabrasion is also useful in the treatment of stretch marks that result from pregnancy.

It is an object of the present invention to provide for a hand tool assembly for use in dermabrasion having an abrasive disc for scrubbing the epidermis outer layers that overcomes at least some of the abovementioned problems or provides the public with a useful alternative.

### SUMMARY OF THE INVENTION

Therefore, in one form of the invention there is proposed, a hand tool assembly for use in dermabrasion including: a body having at least one inlet passage configured to be connected to a vacuum source and including a first opening, said body including a projection; and a cap including a first aperture configured to be removably attached to said handle to thereby form a chamber, the outer edge of said cap includes at least a first and a second recess, said first and second recesses each adapted to engage said projection independently, wherein said second recess is of a greater longitudinally extending depth than said first recess, whereby when said first recess is engaged to said projection said abrasive disc is housed within said chamber and when said second recess is engaged to said projection said abrasive disc protrudes from the chamber through said first aperture; and an abrasive disc configured to be removably attachable to said body, wherein the assembly is adjustable between at least a first position in which the abrasive disc is located within said chamber and a second position in which the abrasive disc protrudes out from within said chamber through said first aperture.

Preferably, the cap is in the form of a hollow dome having a peripheral skirt and includes a first and a second aperture, wherein said second aperture is configured to engage said body and said first aperture is of a diameter larger than the diameter of said abrasive disc, to thereby allow the abrasive disc to pass therethrough.

Preferably, said abrasive disc includes at least one passage therein, said passage being aligned or co-axial with first passage in said body when the abrasive disc is attached thereto.

Preferably, said assembly includes a plurality of abrasive discs of varying diameters, which can independently engage said handle, each said disc having a corresponding cap with an appropriately sized first opening.

Preferably, said body includes an inlet passage and an outlet passage, said inlet passage being connected to a vacuum source and said outlet passage being connected to a particle supply container.

Preferably, said abrasive disc has a first and a second position when attached to said handle, wherein in said second position said abrasive disc extends through said first opening of said cap and in said first position said abrasive disc is housed within said chamber thereby allowing particles to flow through said outlet passage from said supply container.

In a further form of the invention there is proposed, a hand tool assembly for use in dermabrasion including: a body serving as a handle including at least one inlet passage configured to be connected to a vacuum source; an abrasive disc configured to be removably attached to said handle; and at least a first and a second cap, each cap enclosing a respective first and second chamber, so that when the first cap is attached to the body the abrasive disc protrudes from the first chamber and when said second cap is attached to said handle said abrasive disc is housed within the second chamber.

In yet a further form of the invention there is proposed, a hand tool assembly for use in dermabrasion including: a body serving as a handle including at least one inlet passage configured to be connected to a vacuum source; a cap including a first opening, configured to be removably attached to the handle thereby defining a chamber; and an abrasive disc slidably mounted to the handle; wherein the assembly is adjusted between a first position in which the abrasive disc is located within the chamber and a second position in which the abrasive disc protrudes from the chamber through said first opening.

In still yet a further form of the invention there is proposed a hand tool assembly for use in dermabrasion including: a body having at least one inlet configured to be connected to a vacuum source and including a first opening; a cap removably attached to the handle and enclosing a chamber, wherein said cap includes at least a first and a second recess, said first and second recesses each adapted to engage said projection independently; and an abrasive disc removably attachable to said body, wherein the assembly is adjustable between at least a first position in which the abrasive disc is located within the chamber and a second position in which the abrasive disc protrudes from the chamber through said first opening.

Preferably, the second recess is of a greater longitudinally extending depth than the first recess, whereby when the first recess is engaged to the projection the abrasive disc is housed within the chamber and when the second recess is engaged to the projection the abrasive disc protrudes from the chamber through the first opening.

### BRIEF DESCRIPTION OF THE DRAWINGS

The accompanying drawings, which are incorporated in and constitute a part of this specification, illustrate several embodiments of the invention and, together with the description, serve to explain the advantages and principles of the invention. In the drawings,

FIG. 1 is a perspective view of a dermabrasion handpiece illustrating a first embodiment;

FIG. 2A is a side cross-sectional side view of the dermabrasion handpiece of FIG. 1, illustrating a cap in a first position;

FIG. 2B is a side cross-sectional side view of the dermabrasion handpiece of FIG. 1, illustrating a cap in a second position;

FIG. 3A is a side cross-sectional side view of a dermabrasion handpiece, illustrating a second embodiment of the present invention in a first position;

FIG. 3B is a side cross-sectional side view of the dermabrasion handpiece of FIG. 3A in a second position;

FIG. 4 is a perspective view of a dermabrasion handpiece, illustrating a third embodiment;

FIG. 5A is a side cross-sectional side view of the dermabrasion handpiece of FIG. 4 in a first position;

FIG. 5B is a side cross-sectional side view of the dermabrasion handpiece of FIG. 4 in a second position;

FIG. 6 is a perspective view of a dermabrasion handpiece, illustrating a fifth embodiment;

FIG. 7 is a perspective view of a dermabrasion handpiece, illustrating a sixth embodiment;

FIG. 8A is a side view of the dermabrasion handpiece of FIG. 7 in a first position;

FIG. 8B is a side view of the dermabrasion handpiece of FIG. 7 in a second position;

FIG. 8C is a side view of the dermabrasion handpiece of FIG. 7 in a third position;

FIG. 8D is a side view of the dermabrasion handpiece of FIG. 7 in a fourth position;

FIG. 9A is a side cross-sectional side view of the dermabrasion handpiece of FIG. 7 in an extended position;

FIG. 9B is a side cross-sectional side view of the dermabrasion handpiece of FIG. 7 in a retracted position;

FIG. 10A is a side cross-sectional side view of a seventh embodiment of a dermabrasion handpiece in an extended position; and

FIG. 10B is a side cross-sectional side view of the dermabrasion handpiece of FIG. 10A in a retracted position.

### DESCRIPTION OF THE PREFERRED EMBODIMENTS

The following detailed description of the invention refers to the accompanying drawings. Although the description includes exemplary embodiments, other embodiments are possible, and changes may be made to the embodiments described without departing from the spirit and scope of the invention. Wherever possible, the same reference numbers will be used throughout the drawings and the following description to refer to the same and like parts.

The present invention relates to a hand tool assembly used in the abrasion of a skin surface. For a detailed operation of a hand tool for dermabrasion the reader is referred to PCT Publication WO 99/23951 in the name of Ronald Allan Greenberg.

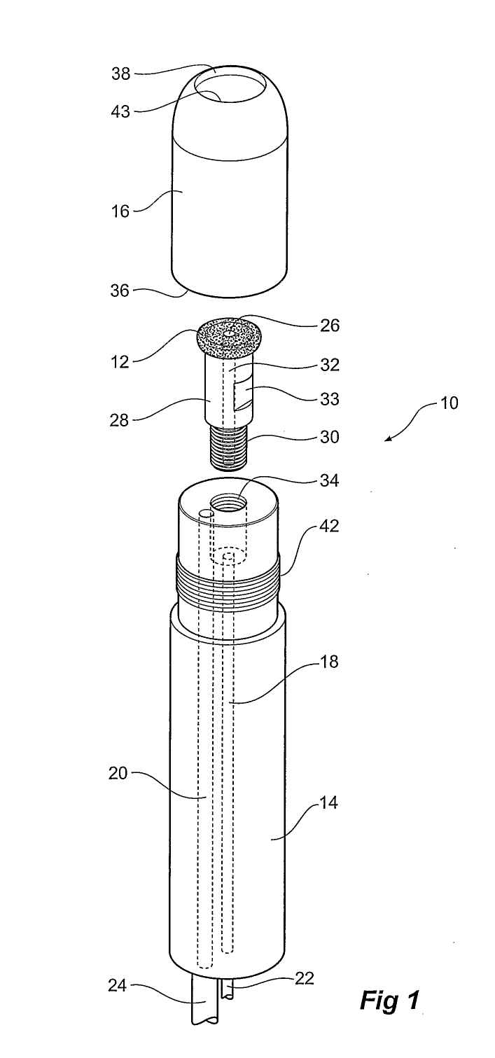

In a first embodiment, as illustrated in FIG. 1, the hand tool assembly 10 includes an abrasive disc 12, a handle 14 and a cap 16. The handle 14 includes an outlet passage 18 and an inlet passage 20. The outlet passage 18 is connected via tube 22 to a particle supply container (not shown) and the inlet passage 20 is connected via a tube 24 to a vacuum source (not shown). The disc 12 which contains an aperture or opening 26 is mounted on a head 28.

The head 28 includes a threaded male member 30 and a longitudinally extending duct 32 which connects to the opening 26. It should be appreciated by the reader that there may be more than one opening 26 in the disc 12 providing all openings 26 connect with the duct 32. Furthermore, the abrasive disc 12 may include its own engagement surface 30 and be connected directly to the handle 14 without the need for a head 28. The head 28 may also include a recess 33 that can be engaged by a tool (not shown) which is used to connect and disconnect the head 28 from the handle 14.

The handle 14 includes an engagement surface or threaded bore 34 that engages a threaded male member 30 enabling the disc 12 to be secured. In this way the abrasive disc 12 can be removably attached to the handle 14 by simply unscrewing. In the present example the engagement surfaces are in the form of threads 30 and 34, however, it should be appreciated that this is only one form of engagement means and another type could be used providing it securely locates the abrasive disc 12 onto the handle 14. The hand tool 10 is configured so that when the head 28 is attached to the handle 14 outlet passage 18 and duct 32 align. This means that during the operation of the hand tool 10 particles are able to flow through the passage 18 into duct 32 and out through opening 26 of the abrasive disc 12.

The present invention provides for different sizes of abrasive discs 12, which can be used for various applications, for instance, the smaller diameter abrasive discs can be used for localized areas such as around the eyes whilst the larger diameter discs can be used on flatter areas of skin. It should, however, be, appreciated by the reader that all sizes of abrasive discs 12 and corresponding caps 16 can be attached to the same handle 14. This therefore provides a cost saving to the user as only one handle 14 has to be purchased. Furthermore, the handle 14 does not need to be completely detached from the vacuum and particle sources since only the abrasive disc 12 and corresponding cap 16 need to be replaced when a different diameter disc 12 is required.

The cap 16 is preferably constructed in the form of a hollow dome which includes a first opening 36 and a second opening 38. An engagement surface 40, as shown in FIGS. 2a and 2b, is located within the chamber of the dome 16 directly adjacent first opening 36. The engagement surface 40 corresponds to an engagement surface 42 on the handle 14. In this way the cap 16 can be removably attached to the handle 14. It should be appreciated that the engagement surface although illustrated in the form as threads is not limited to this particular configuration. The diameter of the second opening 38 is somewhat larger than the diameter of the abrasive disc 12. It is envisaged that the diameter of second opening 38 is around 2-5 mm larger than the abrasive disc 12. However, it should be appreciated that the difference in the diameters is not limited to this particular size range. In this way the abrasive disc 12 can be located in a position where it protrudes through the second opening 38 beyond the surface of the cap 16, or alternatively, it can be located in a position where it is housed within the cap 16 and below the outer rim 43 of the second opening 38.

Turning now to FIG. 2a, the hand tool 10 is shown in first position where the abrasive disc 12 protrudes through the second opening 38. In this arrangement the abrasive disc 12 is applied directly onto the skin and moved to abrade the epidermis 44. The skin cells that are dislodged during this process are drawn up in the direction of arrow 46 through the inlet passage 20 by the vacuum that is produced by the vacuum source (not shown). The abrasive disc 12 allows the user to vigorously abrade the epidermis 44. This is particularly useful where the user is treating scar tissue or blemishes that are deep in the epidermal layer 44.

The cap 16 is then adjusted in the direction of arrow 48 to a second position as illustrated in FIG. 2b. In this position the abrasive disc 12 is contained within the chamber 50 of the cap 16 and does not protrude through the second opening 38. In this arrangement the skin 44 is partially drawn up and seals the chamber 50 and the vacuum produced by the vacuum source (not shown) causes particles to flow from the particle source (not shown) through the passage 18, duct 32 and out through opening 26 of the abrasive disc 12. The particles act to abrade the skin surface 44 and are then drawn up through the inlet passage 20. In this way the skin 44 can be vigorously abraded with the abrasive disc 12 when the cap 16 is in the first position as illustrated in FIG. 2a and then the skin 44 can be gently abraded with the use of particles when the cap 16 is in the second position as illustrated in FIG. 2b. It should be appreciated by the reader that the abrasive disc 12 may be in contact with the skin 44 when the cap 16 is in the second position and thereby may also act to abrade the skin surface 44.

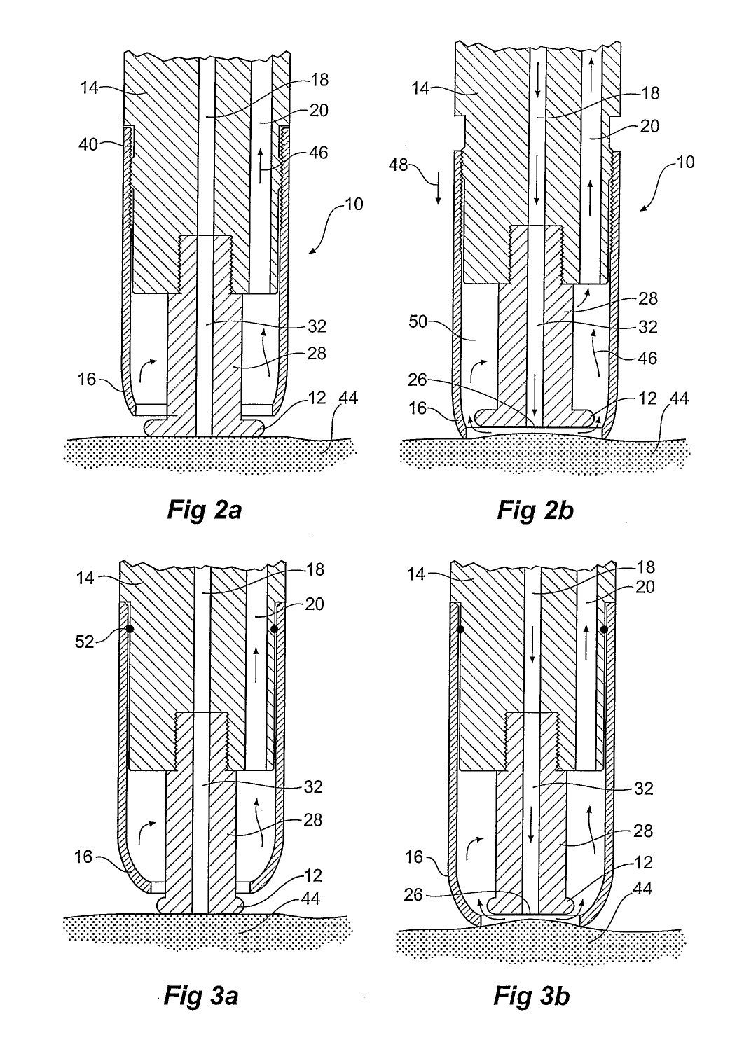

In a further embodiment, as illustrated in FIGS. 3a and 3b, two different sized caps 16 can be used to adjust the position of the abrasive disc 12 in relation to opening 38. In FIG. 3a the cap 16 is of a length that permits the abrasive disc 12 to protrude through the second opening 38 of the cap 16. In contrast, the cap 16 illustrated in FIG. 3b is of a length that ensures that the abrasive disc 12 is contained within the chamber 50 of the cap 16 and does not protrude through opening 38. The caps 16 are frictionally held in place by sealing 0-ring 52.

As illustrated in FIGS. 2a, 2b, 3a and 3b, there are various sizes of abrasive discs 12. However, irrespective of the size of the abrasive disc 12, the distance between the edge of opening 38 and the edge of the disc 12 remains constant. This is accomplished by having caps 16 that correspond to particular sizes of abrasive disc 12.

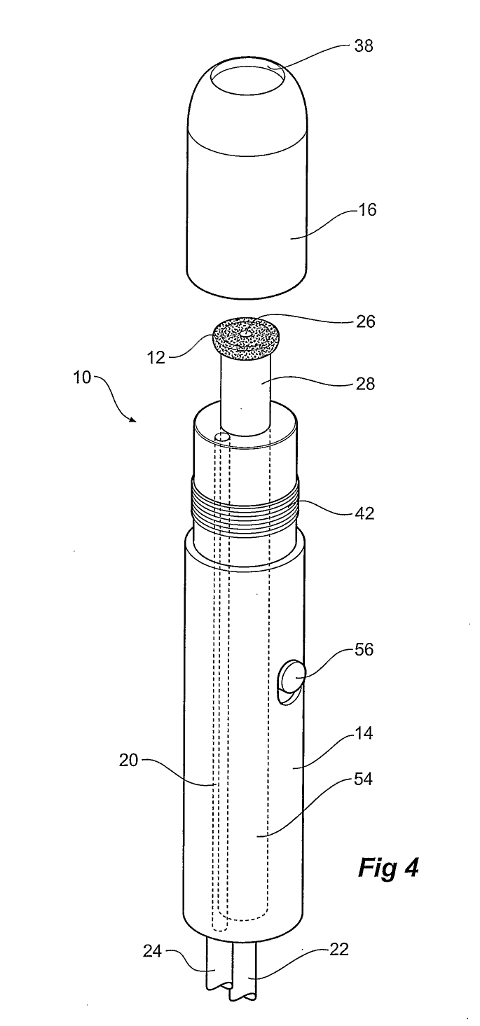

In yet a further embodiment as illustrated in FIG. 4, the head 28 is connected to a shaft 54 which is slidably mounted within the handle 14. In this arrangement the location of the abrasive disc 12 in relation to the opening 38 can be adjusted during operation of the hand tool 10. This avoids the need to disengage the abrasive disc 12 and cap 16 from the handle.

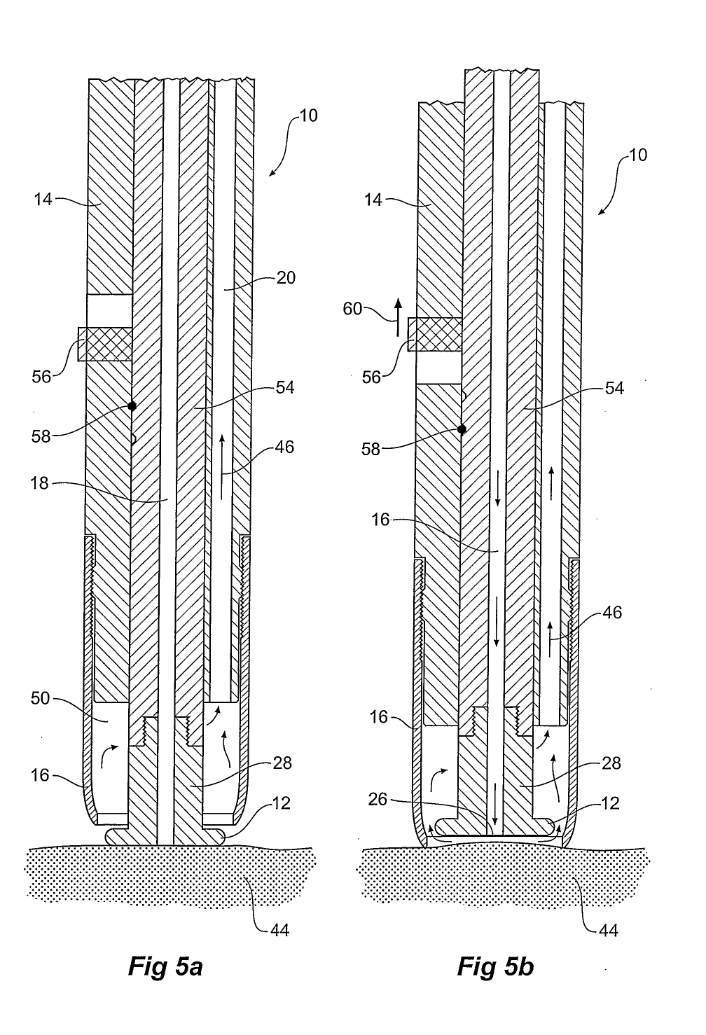

The position of the shaft is adjusted by means of a control switch 56. As illustrated in FIG. 5a, when the switch 56 is in a first position the abrasive disc protrudes through the opening 38. The shaft 54 is located in this position by a locking means 58, which can be in the form of a biased pin. The locking means 58 ensures that maximum pressure can be applied during dermabrasion of the skin 44 without the abrasive disc 12 retracting into the chamber 50 of the cap 16. The switch 56 is then used to move the shaft in the direction of arrow 60 into a second position as illustrated in FIG. 5b. In this position the abrasive disc 12 is contained within the chamber 50 of the cap 16 and particles are able to flow out through the outlet passage 18. The shaft 54 is located in this second position by locking means 58.

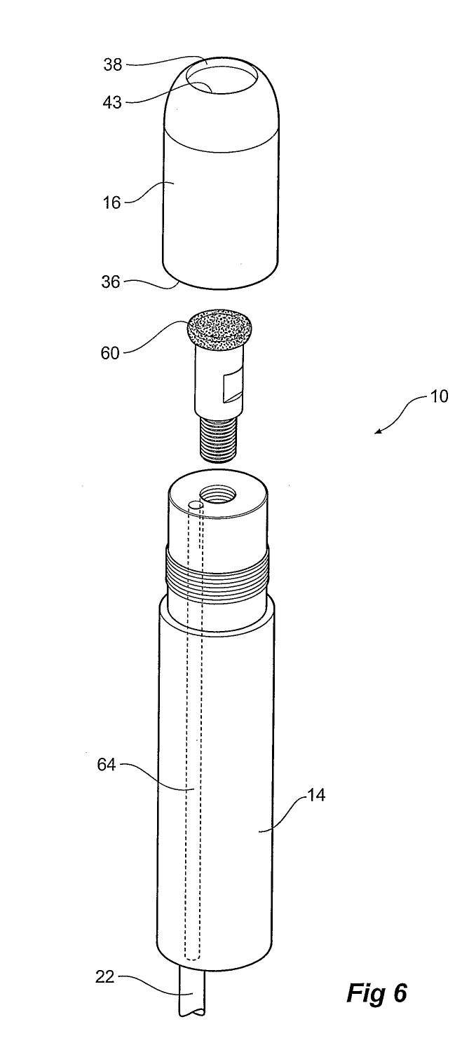

In still a further embodiment as illustrated in FIG. 6, an abrasive disc 60 does not contain an opening. In this form of the invention, the handle contains an inlet 64 that is connected via tube 66 to a vacuum source (not shown). The abrasive disc 60 can be located in a position where it protrudes through the second opening 38 beyond the surface of the cap 16, or alternatively, it can be located in a position where it is housed within the chamber 50 of the cap 16.

When the disc 60 protrudes through opening 38 the epidermis 44 is able to be vigorously abraded by use of the abrasive disc 60. When the disc 60 is in a retracted position and located within the chamber 50 the opening 38 of the hand tool 10 can be brought into contact with the skin 44. The outer rim 43 is then able to create a seal with the skin surface 44. In this way dead skin cells that have been dislodged by the abrasive disc 60 are able to be drawn up through the inlet 64 by the vacuum source. Depending on how far the abrasive disc 60 is retracted it may act to gently abrade the skin 44. This would be particularly useful if the hand tool 10 is being used on sensitive areas such as around the eyes.

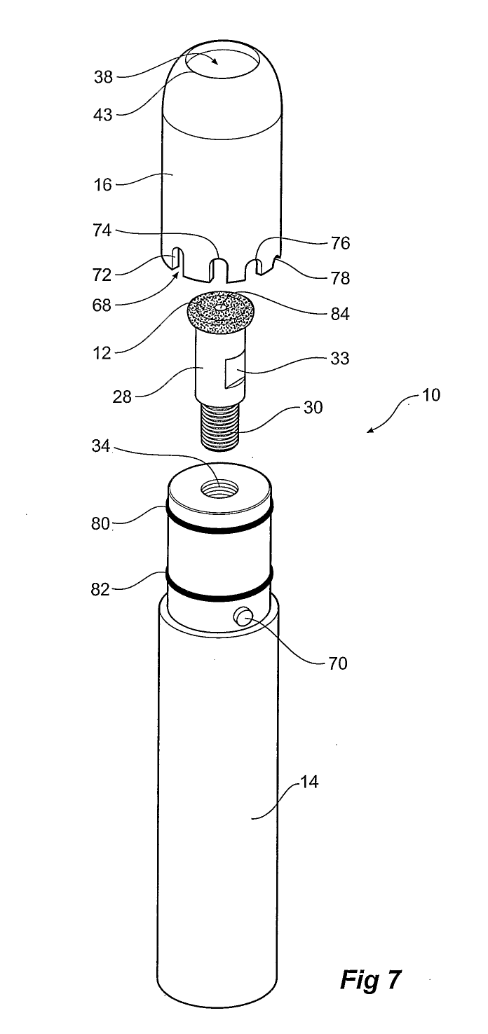

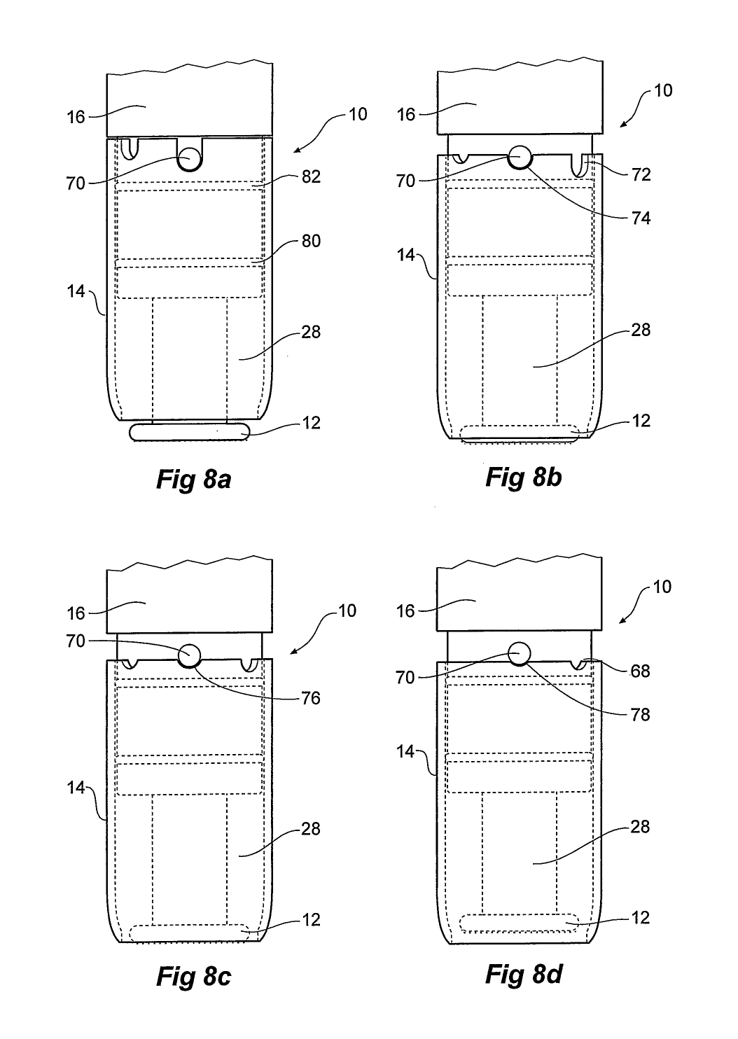

In yet an alternate embodiment, as illustrated in FIG. 7, the cap 16 includes a plurality of deepening recesses 68 which are adapted to engage a projection 70 on handle 14. Four recesses 72, 74, 76 and 78 are illustrated in the figures; however, the invention is not limited to the use of four recesses. The cap 16 slidably engages the handle 14 which includes two 0-rings 80 and 82 which ensure that the only air is drawn in through opening 38. This is to ensure that the greatest amount of suction is produced through opening 38. The head 28, as illustrated in FIG. 7, includes a single inlet 84 in the centre of the abrasive disk 12. The inlet 84 is used to evacuate the loose skin cells from the skin surface by way of a vacuum. As illustrated in FIGS. 8a-8d the recesses 72, 74, 76 and 78 are of different depths, this ensures that the abrasive disk 12 can be located at different locations with respect to opening 38.

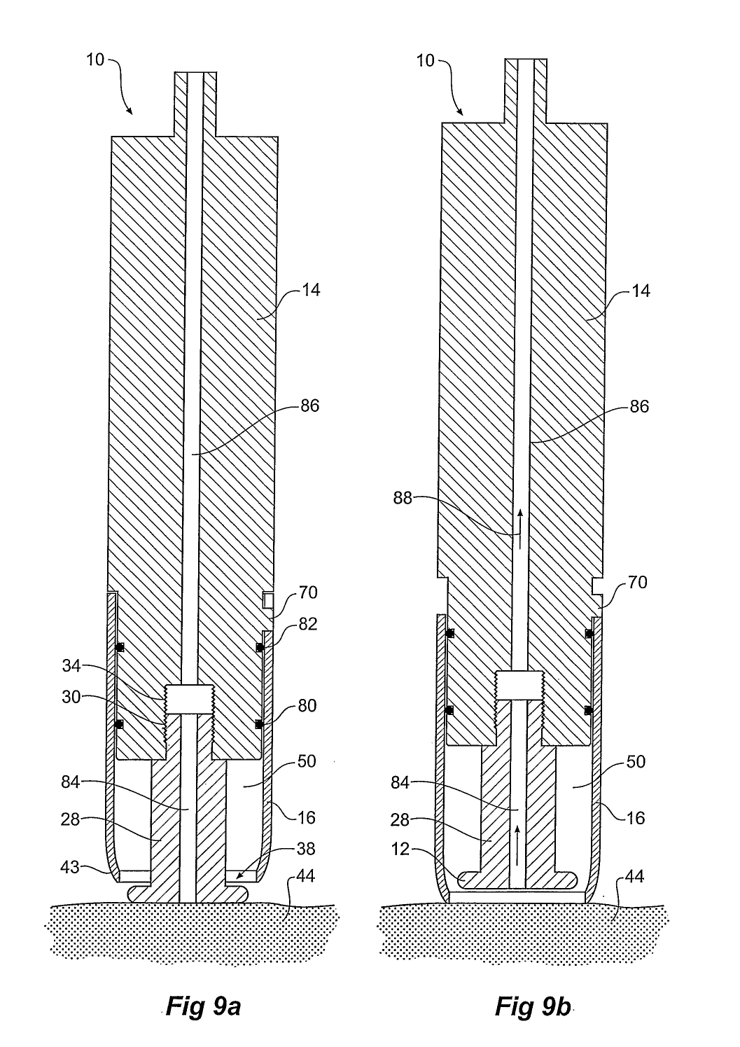

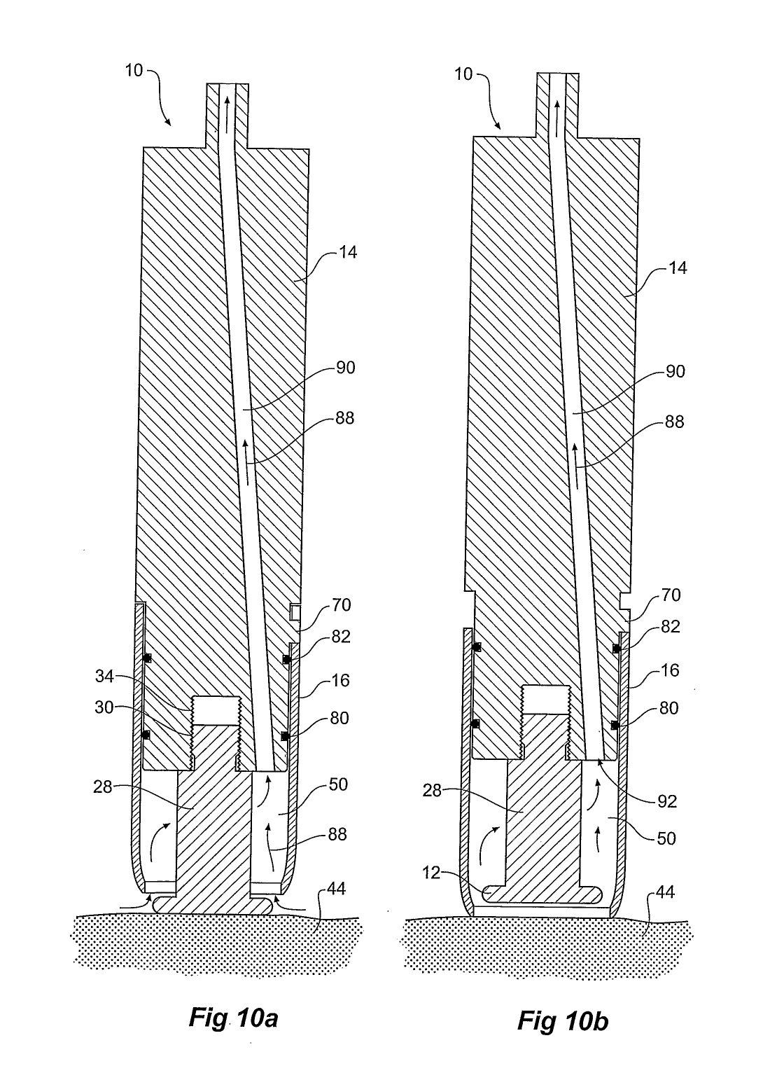

As illustrated in FIG. 9a, when the abrasive disk 12 protrudes from opening 38 the disk 12 is able to be used to abrade the skin 44. When the abrasive disk 12 is withdrawn into chamber 50, as illustrated in FIG. 9b, the vacuum produced by the vacuum source drawn air through duct 86 in the direction of arrow 88. The head 28 is attached to the handle 14 in such a way the duct 86 and inlet 84 align. The vacuum produced within chamber 50 results in loose skin cells being evacuated from the surface of the skin 44 through inlet 84. Alternatively, as illustrated in FIGS. 10a and 10b, the duct 90 can include an opening 92 within chamber 50, which is independent of head 28. In this embodiment, loose skin cells are evacuated through opening 92.

The present invention provides an assembly for abrading the outer layers of skin. The tool of the invention provides the ability to vigorously abrade the skin when the abrasive disc protrudes beyond and through the second opening of the cap. The abrasive disc can then be retracted or repositioned within the chamber enclosed by the cap and the tool can be used to draw off the dead skin particles or gently abraded the skin surface. The tool of the invention has the ability to adjust to the specific requirement of the user as the same handle allows the attachment of variously sized abrasive discs and caps to facilitate treatment. The use of a universal handle that can engage all the different sizes and configurations of abrasive discs reduces the cost of the apparatus and facilitates ease of use.

Further advantages and improvements may very well be made to the present invention without deviating from its scope. Although the invention has been shown and described in what is conceived to be the most practical and preferred embodiment, it is recognized that departures may be made therefrom within the scope and spirit of the invention, which is not to be limited to the details disclosed herein but is to be accorded the full scope of the claims so as to embrace any and all equivalent devices and apparatus.

1. A hand tool assembly for use in dermabrasion, comprising:
a body having at least one inlet passage configured to be connected to a vacuum source, said body including a first opening and at least one projection;
an abrasive disc configured to be removably attached to said body; and
a cap configured to be removably attached to said body to thereby form a chamber, said cap having an outer edge and a first aperture, said outer edge of said cap including at least first and second recesses, said first and second recesses being adapted to independently engage said projection, said second recess having a greater longitudinally extending depth than said first recess, whereby when said first recess is engaged to said projection said abrasive disc is housed within said chamber and when said second recess is engaged to said projection said abrasive disc protrudes from said chamber through said first aperture;
said assembly being adjustable between at least a first position, wherein said abrasive disc is located within said chamber, and a second position, wherein said abrasive disc protrudes out from within said chamber through said first aperture.

2. The assembly of claim 1, wherein said cap is in the form of a hollow dome having a peripheral skirt, said peripheral skirt including a first and a second aperture, wherein said second aperture is configured to engage said body and said first aperture has a larger diameter than the diameter of said abrasive disc, to thereby allow said abrasive disc to pass therethrough.

3. The assembly of claim 1, wherein said abrasive disc includes at least one passage therein, said passage being aligned or co-axial with said first passage in said body when said abrasive disc is attached thereto.

4. The assembly of claim 1, wherein said assembly includes a plurality of abrasive discs that are adapted to independently engage said handle, said abrasive discs having varying diameters, each of said abrasive discs having a corresponding cap that includes a first opening.

5. The assembly of claim 1, wherein said body includes an inlet passage and an outlet passage, said inlet passage being configured to be connected to a vacuum source and said outlet passage being configured to be connected to a particle supply container.

6. The assembly of claim 5, wherein said abrasive disc has a first and a second position when attached to said handle, wherein in said second position said abrasive disc extends through said first opening of said cap and in said first position said abrasive disc is housed within said chamber thereby allowing particles to flow through said outlet passage from said particle supply container.

7. A hand tool assembly for use in dermabrasion, comprising:
a body serving as a handle having at least one inlet passage configured to be connected to a vacuum source;
an abrasive disc configured to be removably attached to said handle; and
at least a first and a second cap, each of said caps enclosing a respective first and second chamber, whereby when said first cap is attached to said body said abrasive disc protrudes from said first chamber and when said second cap is attached to said handle said abrasive disc is housed within said second chamber.

8. A hand tool assembly for use in dermabrasion, comprising:
a body serving as a handle having at least one inlet passage configured to be connected to a vacuum source;
a cap configured to be removably attached to said handle thereby defining a chamber, said cap including a first opening;
an abrasive disc slidably mounted to said handle;
wherein said assembly is adjustable between a first position, wherein said abrasive disc is located within said chamber, and a second position wherein said abrasive disc protrudes from said chamber through said first opening.

9. A hand tool assembly for use in dermabrasion, comprising:
a body having at least one inlet configured to be connected to a vacuum source, said body including at least one projection and a first opening;
a cap removably attached to said body and enclosing a chamber, wherein said cap includes at least a first and a second recess, said first and second recesses each adapted to independently engage said projection; and
an abrasive disc removably attachable to said body, wherein said assembly is adjustable between at least a first position, wherein said abrasive disc is located within said chamber and a second position, wherein said abrasive disc protrudes from said chamber through said first opening.

10. The assembly claim 9, wherein said second recess has a greater longitudinally extending depth than said first recess, whereby when said first recess is engaged to said projection said abrasive disc is housed within said chamber and when said second recess is engaged to said projection said abrasive disc protrudes from said chamber through said first opening.
